(19) United States
(12) Patent Application Publication
(10) Pub. No.: US 2018/0200446 A1
(43) Pub. Date: Jul. 19, 2018

(54) AUTOMATIC INJECTION DEVICE

(71) Applicant: OWEN MUMFORD LIMITED, Oxfordshire (GB)

(72) Inventors: James Paul GRIMOLDBY, Woodstock (GB); Oliver Gareth HYDE, Woodstock (GB)

(21) Appl. No.: 15/744,259

(22) PCT Filed: Jul. 19, 2016
(86) PCT No.: PCT/GB2016/052174

§ 371 Date: Jan. 12, 2018

(30) Foreign Application Priority Data
Aug. 13, 2015 (GB) 1514383.7

(51) Int. Cl. A61M 5/315; A61M 5/20; A61M 5/32

(52) U.S. Cl. CPC A61M 5/31501; A61M 5/2033; A61M 5/31553; A61M 2005/2086; A61M 5/3204; A61M 2005/206; A61M 2005/2073; A61M 5/3156

(57) ABSTRACT

There is provided an injection device for delivering a medicament from a container. The device includes, a housing for housing a container, a plunger substantially housed within the housing and which is movable within the housing, a force applicator for applying a force to the plunger, a trigger coupled to the force applicator for releasing the force applicator to fire the device, a boot, a dose selector for allowing a user to select a dose of medicament, and a first mechanical interlock arranged such that the force applicator cannot be released until the dose selector has been operated to select the dose of medicament, and a second mechanical interlock arranged such that the dose selector cannot be operated to select the dose of medicament prior to removal of the boot.

### FIELD OF THE INVENTION

The present invention relates to injection devices for delivering a medicament from a container.

### BACKGROUND OF THE INVENTION

Injection devices, such as automatic injection devices, are routinely used in the medical field to deliver a measured dose of medicine to a user. Typically, injection devices have a user friendly design, allowing them to be safely used by patients for self-administration, although in some circumstances they may be used by trained personnel. They may be designed to be carried by the user for use at any time, in which case they should be as small and inconspicuous as possible to improve user compliance. Automatic injection devices for the self-administration of parenteral drugs include single dose and multi dose reusable and disposable auto-injectors and pen injectors (e.g. insulin pens), which are suitable for a wide range of primary containers, including pre-filled glass and plastic syringes and pre-filled cartridges.

A typical automatic injection device comprises several parts which may include; a syringe containing medicine, a needle fixed to the end of the syringe, a firing mechanism including a spring (or possibly other drive means such as an electric motor or gas drive means), a trigger, and a dose selector which allows a user to select a dose of medicine that they require. The firing mechanism is activated by the trigger and forces the medicine through the needle and into the user. The firing mechanism may also be arranged to perform an initial step of inserting the needle through the skin using the force provided by the injection spring (or possible a secondary spring). A mechanical lock may be provided to prevent the trigger from being accidentally pressed. This could be, for example, a catch that must be moved out of the way in order to access the trigger.

Automatic injection devices are delivered to end users in an assembled state, with a medicine syringe contained within the device housing and a needle fixed to the end of the syringe. In order to ensure sterility of the needle, the projecting end of the needle is contained within a rubber, elastomer “boot”, which may have a rigid polymer cover. Typically, the boot forms an interference fit around the narrowed end portion of the syringe housing. The tip of the needle may penetrate the end of the boot.

The injection device may also comprise a boot remover to allow the end user to easily and safely remove the boot and thereby expose the needle. Typically, the boot remover is fitted around or inside a proximal end (end closest to injection site) of the device prior to insertion of the syringe into the housing. A needle shield may be further provided around the needle, such that the needle remains protected even after the boot has been removed. This is relevant to automatic injection devices which, in addition to driving the medicine through the needle (medicine delivery phase), perform the initial step of inserting the needle through the skin (needle insertion phase).

When an automatic injection device is to be used, typically a user removes the boot using the boot remover to expose the needle, and then selects a dose of medicine to be delivered. The user will then release the mechanical lock, such that the trigger can be pressed, place the automatic injection device against the surface of the skin and press the trigger to push the needle through the skin and force the medicine through the needle. A carriage and carriage-return spring may cause the needle to be returned to a position within the needle shield to prevent accidental injury after the device has been used.

A problem with injection devices occurs when a user forgets to first remove the boot, and, instead, operates the trigger with the boot still in place. If the boot is not removed before firing, no drug is delivered to the user. Furthermore, since the medicine will now be under pressure, there is a risk that the user may inadvertently empty the syringe contents into the air if, when realising their error, they subsequently remove the boot.

A user may not have an abundance of medicine and so waste may be a serious issue. Waste may also be undesirable due to cost implications: some medicines can be extremely expensive. Therefore, there exists a need to provide an improved automatic injection device.

### SUMMARY

In a first aspect of the invention, there is provided an injection device for delivering a medicament from a container. The device comprises, a housing for housing a container, a plunger substantially housed within the housing and which is movable within the housing, a force applicator for applying a force to the plunger, a trigger coupled to the force applicator for releasing the force applicator to fire the device, a boot, a dose selector for allowing a user to select a dose of medicament, and a first mechanical interlock arranged such that the force applicator cannot be released until the dose selector has been operated to select the dose of medicament, and a second mechanical interlock arranged such that the dose selector cannot be operated to select the dose of medicament prior to removal of the boot.

The two mechanical interlocks force the user to perform a sequential order of steps before the injection device will fire. Advantageously, this prevents a user from accidentally firing the device while the boot is still attached, or while no dose is set.

The force applicator may be a helical spring which, in an initial, unfired, condition is held in a compressed state. The trigger may not physically contact the force applicator, but just be linked to the force applicator such that on activating the trigger (such as by pressing it), the helical spring is no longer held in the compressed state, and is able to expand so as to deliver medicament. The term, fire, may refer to any action involved with delivering the medicament. For example, when the device is fired, the needle may be driven into a user's skin (needle insertion phase) followed by the medicament being forced through the needle and into the user (medicament delivery phase).

The first mechanical interlock may be provided by a first coupling between the housing and the plunger. The first coupling may comprise an abutment between a first abutment element on, or coupled to, one of the housing and the plunger and a second abutment element on, or coupled to, the other of the housing and the plunger.

The term “coupled” is used to denote that the components are mechanically linked, such that a force applied to one component ultimately causes a force to be applied to the other component. For example, the abutment need not be between features on the plunger and housing, but may, for example, be between features of components coupled to the plunger and housing, such as intermediate components between the plunger and housing.

The first mechanical interlock may be arranged such that the abutment of the first abutment element and the second abutment element prevents the plunger from being displaced axially. For example, the first coupling may comprise an abutment between a shoulder on the housing and a peg on the plunger, preventing proximal axial movement of the plunger. In order to fire the device, the peg may need to be moved such that it does not abut the shoulder.

The second mechanical interlock may be provided by a second coupling between the dose selector and the boot. The second coupling may comprise an abutment between a third abutment element on, or coupled to, the dose selector and a fourth abutment element on, or coupled to, the boot. For example, the third abutment element and fourth abutment element need not be features on the dose selector and boot, but may, for example, be features on components coupled to the dose selector and boot, such as intermediate components between the dose selector and boot.

The dose selector may be rotatable relative to the housing, said rotation allowing a user to select the dose of medicament. The dose selector and plunger may be rotationally coupled such that rotation of the dose selector rotates the plunger and removes the abutment of the first abutment element and the second abutment element.

Optionally, the first abutment element may be either a peg or shoulder on the plunger and the second abutment element may be the other of a peg or shoulder on the housing. For example, the first coupling may comprise an abutment between a shoulder on the housing and a peg on the plunger, preventing axial movement of the plunger. Upon rotation of the dose selector, the plunger is rotated, which displaces the peg relative to the shoulder such that the peg no longer abuts the shoulder, allowing the plunger to be proximally axially displaced.

The second mechanical interlock may be arranged such that the abutment of the third abutment element and the fourth abutment element prevents the dose selector from being rotated relative to the housing. For example, the abutment of the third abutment element and the fourth abutment element may prevent a user from setting a dose using the dose selector.

Optionally, removal of the boot may remove the second coupling between the third abutment element and the fourth abutment element, allowing rotation of the dose selector with respect to the housing.

The third abutment element may be a first surface on the plunger and the fourth abutment element may be a second surface coupled to the boot. If the plunger is coupled to the dose selector, such that they rotate together, then preventing the plunger from rotation will prevent the dose selector from rotation. The first surface may be a protrusion, such as a peg, on the plunger, and the second surface coupled to the boot may be a part of a boot remover, or may be a part of an internal sleeve rotationally fixed and axially moveable with respect to the housing, boot and/or boot remover.

The injection device may further comprise a sleeve housed in the housing and which may be rotationally fixed and axially moveable with respect to the housing. The sleeve may take the form of a tube, or partial tube, that fits within the housing. The sleeve may comprise the second surface and the sleeve may be axially coupled to the boot such that axially movement of the sleeve is restricted while the boot is attached to the device. The second surface may axially extend past the first surface such that the second surface presents a barrier to rotation of the first surface.

The injection device may further comprise a boot remover for removing the boot. The axial coupling between the sleeve and boot may act between a boot remover, where the boot remover may abut the sleeve while the boot remover is attached to the device, preventing axial movement of the sleeve.

The first abutment element and the third abutment element may be the same abutment element. For example, the first abutment element and the third abutment element may be the same peg on the plunger.

When the device is fired, the plunger may be arranged to abut the sleeve so as to axially displace the sleeve. For example, as the plunger is axially displaced in a proximal direction (towards the user's skin), the plunger may also proximally axially displace the sleeve. The abutment between the plunger and the sleeve may be via the first and/or third abutment element abutting a distal surface of the sleeve. For example, a peg of the plunger may abut a distal end of the sleeve during displacement.

The housing may comprise a viewing window, and the sleeve may be arranged such that a portion of the sleeve is visible through the viewing window after the device has been fired. Advantageously, this provides a visual cue to the user that the device has been fired. Alternatively, a portion of the sleeve may be visible prior to firing the device, and during a firing process the sleeve is displaced such that a portion of the sleeve is not visible through the viewing window after firing the device.

The sleeve may comprise a step like profile along its distal end, where at least one step corresponds with a particular dose. The steps may provide the distal surface that the first and/or third abutment element abut on the sleeve. For example, if the first and third abutment element is a peg on the plunger, the peg abuts a step corresponding to the selected dose during firing of the device, so as to axially displace the sleeve.

The sleeve may further comprise a resiliently flexible arm having a wedge portion, and may be arranged such that, prior to firing the device, the resiliently flexible arm is bent radially inward due to an abutment between the wedge portion and an inner surface of the housing, placing the resiliently flexible lock arm under tension. The resiliently flexible arm may further be arranged such that after firing the injection device, the wedge portion is proximally displaced so as to line up with the viewing window such that the wedge portion no longer abuts the inner surface of the housing, allowing the tension in the resiliently flexible lock arm to be released, driving the wedge portion into the viewing window.

In a second aspect of the invention there is provided an injection device for delivering a medicament from a container. The device comprises a housing for a container, a plunger movable within the housing to expel a dose of medicament, a force applicator for applying a force to the plunger, a trigger coupled to the force applicator for releasing the force applicator, a dose selector for allowing a user to select a dose of medicament from a plurality of doses, and an indicator element for indicating to a user that a selected dose has been delivered, the indicator element being arranged to be axially moveable by the plunger from a first position when a selected dose of medicament has not been expelled, to a second position when a selected dose of medicament has been expelled, and wherein the plunger and the indicator element are arranged such that the axial distance traveled by the indicator element between the first and second position is substantially the same for each of the plurality of doses.

Advantageously, the second aspect provides an injection device that can be set to deliver a large range of doses of medicament, while reliably providing an indication that the selected dose has been delivered. This is due, in part, to the fact that the indicator element is driven proximally forward by the plunger for the same distance, regardless of what dose is set, and therefore what distance the plunger travels.

The indicator element may be substantially housed in the housing and may comprise a sleeve portion. The sleeve portion may take the form of a tube, or partial tube, that fits within the housing. The sleeve portion may comprise a step like profile along a part of its distal end, wherein each step corresponds with a specific dose of medicament, and defines an axial distance which the plunger must travel before a peg of the plunger makes contact with the step and axially displaces the indicator element from the first position to the second position.

The housing may comprise a plurality of tracks, where each track corresponds to a specific dose and has a corresponding length associated with a specific dose. For example, longer tracks correspond with larger doses, and shorter tracks with smaller doses. The tracks may be arranged to receive the peg of the plunger. In this way, the tracks define how far the plunger may travel, and therefore define the amount of medicament expelled. The specific steps of the step like profile may be arranged to correspond with specific track lengths such that the axial distance traveled by the indicator element between the first and second position is substantially the same for each of the plurality of doses.

The relative change in height of each step may directly correspond with the relative change in the length of each track. For example, longer tracks may correspond with steps that define a shorter length of the sleeve portion, and shorter tracks may correspond with steps that define a longer length of the sleeve portion.

The housing may further comprise a viewing window. The indicator element may comprise a visual indicator which is arranged to line up with the viewing window when in the second position, so as to indicate to a user that a selected dose has been delivered.

The visual indicator may comprise a wedge portion coupled to a resiliently flexible arm, and may be arranged such that, prior to firing the device, the resiliently flexible arm is bent radially inward due to an abutment between the wedge portion and an inner surface of the housing, placing the resiliently flexible lock arm under tension. The resiliently flexible arm may further be arranged such that after firing the injection device, the wedge portion is proximally displaced so as to line up with the viewing window such that the wedge portion no longer abuts the inner surface of the housing, allowing the tension in the resiliently flexible lock arm to be released, driving the wedge portion into the viewing window.

In a third aspect of the invention, there is provided a plunger for use in an injection device. The plunger comprises a first portion and a second portion, the first and second portions being formed as separate components, and wherein the first portion is arranged to receive and accommodate the second portion in one of a plurality of positions, wherein each position defines a specific length of the plunger.

The first portion may be a distal portion of the plunger, and the second portion may be a proximal portion of the plunger. Advantageously, the third aspect provides a plunger that's length can easily be adjusted during assembly by altering the position at which the two portions of the plunger are assembled. This allows fixed size components to be manufactured, which can then be combined to achieve a plunger having a range of possible lengths. The lengths may relate to the specific doses that the injection device, in which the plunger is to be used, delivers.

The first portion may comprise an opening arranged to receive the second portion.

The opening may comprise a recessed region along an edge of the first potion. The recessed region may comprise a series of saw tooth features which may be arranged to interlock with corresponding saw tooth features on the second portion.

Alternatively, the opening may comprise an opening on a proximal end of the first portion, and may be arranged such that a distal portion of the second portion may be loaded into the opening. The first portion may comprise a plurality of apertures along an axial length of the first portion, each aperture defining a particular length of the plunger, and the second plunger potion may comprises an extendable arm which is arranged to enter one of the apertures in the first plunger portion so as to hold the second plunger portion in place relative to the first plunger portion.

In a fourth aspect of the invention, there is provided a method of manufacturing a plunger. The method comprises, forming a first portion having means to receive and accommodate a second portion in one of a plurality of positions, wherein each position defines a specific length of the plunger, forming a second portion, and accommodating the second portion in a position of the plurality of positions.

In a fifth aspect of the invention, there is provided an injection device for delivering a medicament from a syringe. The device comprises, a housing for housing a syringe, a plunger substantially housed within the housing and which is movable within the housing, a force applicator for applying a force to the plunger, a trigger coupled to the force applicator for releasing the force applicator, and a high friction surface coupled between the plunger and the housing, and arranged to reduce the initial acceleration of the plunger while the force is applied to the plunger during a needle insertion phase.

The high friction surface is a surface having a relatively high coefficient of friction compared with other materials typically used in an injection device. For example, the high friction surface may be provided by a rubber material. The high friction surface may be applied to the housing, and the plunger may be arranged to slide against the rubber material. The high friction surface may be applied to any component of the injection device that the plunger axially moves relative to during a needle insertion phase.

Typically, a force applicator, such as a helical spring, performs the job of inserting a needle and displacing a bung in a syringe so as to deliver medicament. This can lead to peak impacts which are absorbed by components of the syringe such as a flange of the syringe. Such peak impacts can damage components of the syringe and/or injection device. By providing a high friction surface, the initial acceleration of the plunger is reduced, thereby reducing the magnitude of the peak impacts.

The plunger or housing may comprise the high friction surface and the other of the plunger or housing may comprise a surface which is arranged to slide against the high friction surface so as to reduce the initial acceleration of the plunger.

The high friction surface may have an axial length that corresponds to a length traveled by the plunger during the needle insertion phase of the injection device, such that the high friction surface reduces the acceleration of the plunger during the needle insertion phase. Once the needle has been inserted, the plunger clears the high friction surface, allowing the medicament to be delivered with the force applicator being undamped.

The housing may further comprise tracks of differing lengths, each track corresponding to a specific dose, the tracks being arranged to receive and accommodate a peg coupled to the plunger, and wherein the tracks comprise the high friction surface or a further high friction surface. The peg of the plunger may then be arranged to slide against the high friction surface applied to the track, reducing the initial acceleration of the plunger.

The plunger may comprise a bore which is arranged to accommodate a rod which is axially fixed with respect to the housing, and the rod may comprise the high friction surface or a further high friction surface. In an unfired position, the rod will be located within the bore, and an interference fit is achieved between the high friction surface on the rod and the inner surface of the bore. As the injection device is fired, the plunger moves axially with respect to the rod, meaning that the inner surface of the bore slides against the high friction surface on the rod, reducing the initial acceleration of the plunger.

The injection device may further comprises a syringe carrier arranged to accommodate a syringe, wherein the syringe carrier may be axially displaced during the needle insertion phase, a resiliently deformable material arranged between the syringe carrier and a syringe, wherein the resiliently deformable material may be arranged such that when the syringe carrier reaches the end of its travel, axial movement between the syringe carrier and a syringe is damped by the resiliently deformable material.

The resiliently deformable material may be arranged to act between a flange of the syringe carrier and a flange of a syringe. The resiliently deformable material may be in the form of a lip around a distal end of the syringe carrier. As the distance between the syringe flange and the syringe carrier flange reduces, the resiliently deformable material is compressed between the two flanges, absorbing energy and reducing the impact between the flanges.

The high friction surface and/or the resiliently deformable material may comprise a thermoplastic elastomer.

### BRIEF DESCRIPTION OF DRAWINGS

FIG. 1 is a perspective view of an injector device;

FIG. 2a is a cross-section of the injector device;

FIG. 2b is a cross-section of the injector device along a plane perpendicular to the cross section of FIG. 2a;

FIG. 3 is a perspective view of a dose selector of the injector device;

FIG. 4 is a perspective view of a distal end of a housing;

FIG. 5 is a perspective view of a lock shuttle and trigger catch of a plunger in isolation;

FIG. 6 is a further perspective view of the lock shuttle and trigger catch;

FIG. 7 is a perspective partial view through the dose selector and showing a distal part of the lock shuttle;

FIG. 8 is a perspective partial view of the proximal end of the dose selector;

FIG. 9 is a side on view of a shuttle carrier in isolation;

FIG. 10 is a perspective cross-section view of a distal end of the housing, showing the trigger catch within the housing;

FIG. 11 is a perspective view of the lock shuttle and boot remover in isolation;

FIG. 12a is a partial see-through view of the injection device showing the trigger catch in a mid-delivery position;

FIG. 12b is a close up external view of the housing showing a viewing window of the injection device shown in FIG. 12a;

FIG. 13a is a partial see through view of the injection device showing the trigger catch following delivery of a dose of medicament (fired position);

FIG. 13b is a close up external view of the housing showing a viewing window of the injection device shown in FIG. 13a;

FIG. 14 is a cross section of a perspective view of the injection device, showing the relative position of steps on the lock shuttle with respect to tracks within the housing;

FIG. 15 is a close up side on view of a peg of the plunger;

FIG. 16 shows two perspective views of a plunger in an assembled, and unassembled state.

FIG. 17 shows two perspective views of an alternative plunger in an assembled, and unassembled state.

FIG. 18 is a close up cross sectional view of a distal end of a syringe;

FIG. 19 is a close up cross sectional view of the trigger lock prior to firing the injection device;

FIG. 20 is a close up cross sectional view of the trigger lock during firing of the injection device;

FIG. 21 is a perspective, cross sectional view showing the plunger and inner surface of the housing;

FIG. 22 is a cross sectional view of a distal end of the injection device having a modified plunger; and

FIG. 23 is a perspective, cross sectional view of the injection device shown in FIG. 22.

### DETAILED DESCRIPTION

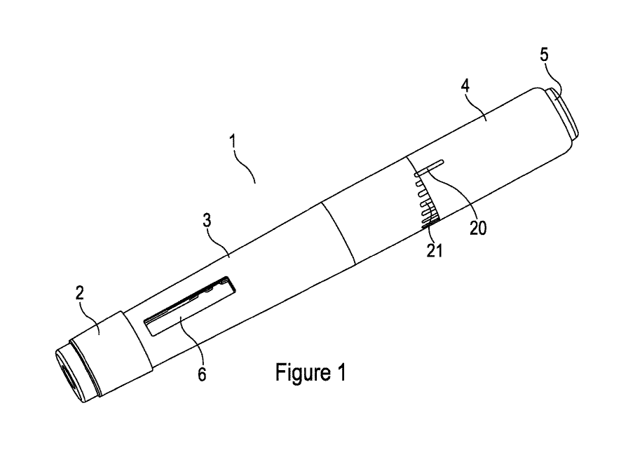

With reference to FIGS. 1 to 15, there will now be described an injection device 1 according to the invention. The embodiment shown is a specific embodiment and is not intend to limit the manner in which the invention is implemented. For example, where some parts are shown as separate but would function mechanically if they were integral then the skilled person will recognise that both implementations are disclosed herein; similarly where certain parts are shown as integral but could be provided as two or more parts then such an implementation is also envisaged as falling within the present invention.

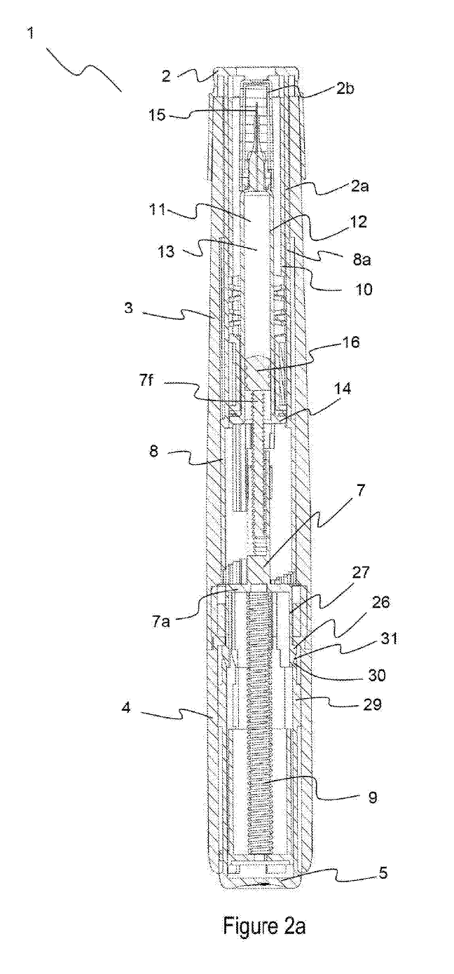

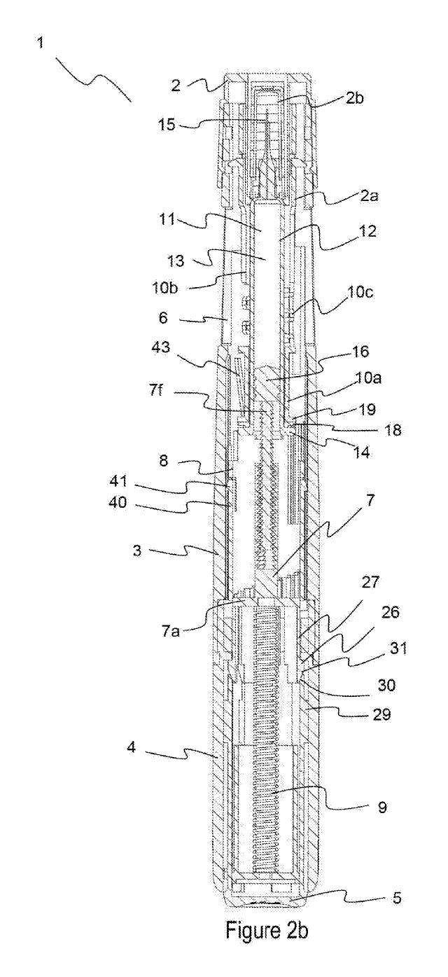

FIG. 1 shows a perspective view of an automatic injection device 1, henceforth referred to as an auto-injector 1, showing, amongst other components, a boot remover 2, a housing 3, a dose selector 4, a trigger button 5 and a viewing window 6. FIGS. 2a and 2b show cross sections through the auto-injector 1, (where the plane of the cross section of FIG. 2a is at 90 degrees of the plane of the cross section of FIG. 2b) showing, amongst other components, a boot 2b, a plunger 7, a lock shuttle 8, a drive spring 9, a syringe carrier 10 and a syringe 11.

The terms distal and proximal are sometimes used interchangeably. In the following description, the distal direction refers to a direction away from an injection site and the proximal direction refers to the direction towards the injection site. Therefore, the trigger button 5 is located at a distal end of the auto-injector 1 and the boot remover 2 is located at a proximal end of the auto-injector 1.

The syringe 11 comprises a generally cylindrical container portion 12 for accommodating a fluid 13 such as a medicament, syringe flange 14, and a needle 15. The needle 15 is in communication with the interior of container portion 12 so that the fluid 13 may be expelled through needle 15. A bung 16 is inserted in the container portion 12 at the distal end of the container portion 12. The bung 16 seals the fluid 13 within the container portion 12 and is arranged such that proximal displacement of the bung 16, relative to the container portion 12, expels the fluid 13 through the needle 15.

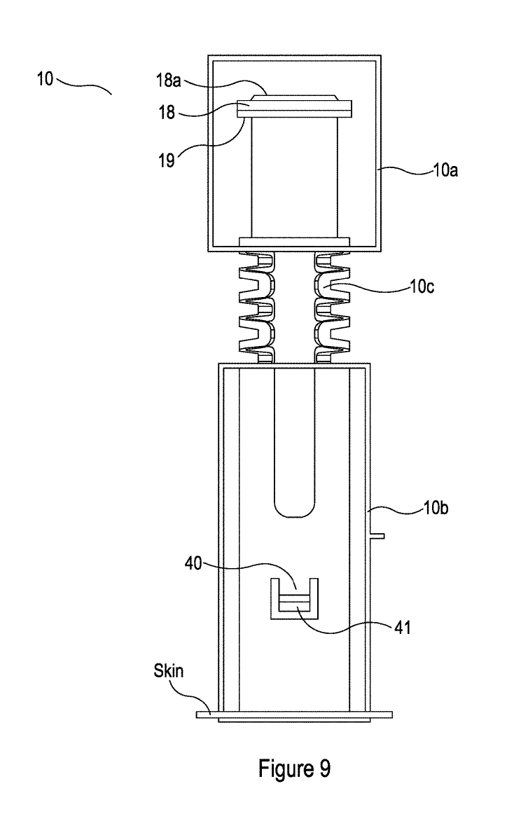

The syringe carrier 10 houses the syringe 11 (or other container for a substance). The syringe carrier 10 comprises a distal barrel portion 10a, a proximal barrel portion 10b and a compressive connector portion 10c (see FIG. 9). In the embodiment shown in the figures, the compressive connector portion 10c comprises an integral spring like structure which is arranged between the distal barrel portion 10a and the proximal barrel portion 10b. When the compressive connector portion 10c is in an uncompressed state, the proximal barrel portion 10b of the syringe carrier 10 extends beyond the needle 15 so as to provide a shield for the needle 15. When the compressive connector portion 10c is in a compressed state, the proximal barrel portion 10b of the syringe carrier 10 does not extend beyond the needle 15, exposing the needle 15.

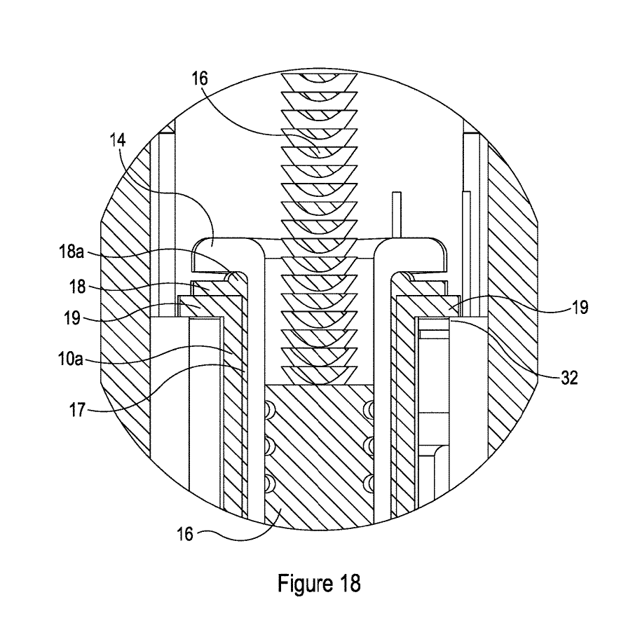

The syringe 11 is substantially axially held relative to the syringe carrier 10 by an interference fit between the syringe 11 and the distal barrel portion 10a of the syringe carrier 10. A rubber sleeve 17 is positioned between the syringe 11 and syringe carrier 10 to help provide the interference fit (see FIG. 18 and further description below). However, during an injection process, the forces involved may overcome the interference fit, causing the syringe 11 to move proximally relative to the syringe carrier 10 (described in detail below). A resiliently deformable flange damper 18 is located between the syringe flange 14 and a flange 19 on the distal barrel portion 10a of the syringe carrier 10 (see FIG. 18) in order to help reduce the load on the syringe flange 14 during an injection process. In the embodiment shown in the FIG. 18, the rubber sleeve 17 and resiliently deformable flange damper 18 are integral. However, the skilled person will realise that these components may be separate.

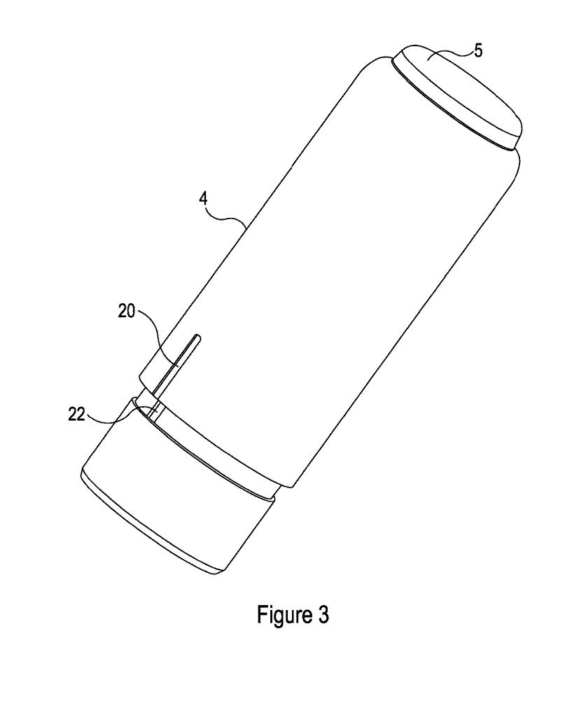

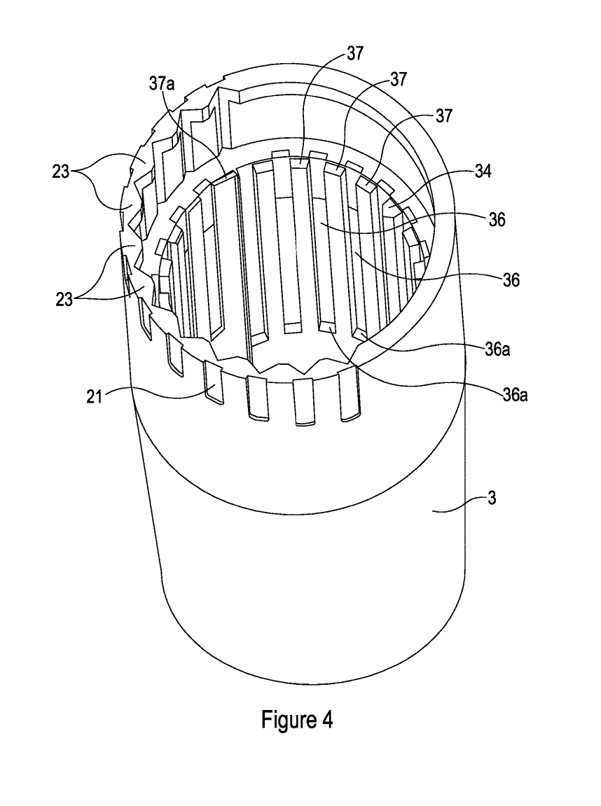

The auto-injector 1 is arranged such that a user may select discrete doses to be expelled from the syringe 11 by rotating the dose selector 4, in this case, anticlockwise relative to the housing 3. The dose selector 4 is prevented from initially rotating clockwise via an abutment of features between the dose selector 4 and the housing 3 (described below). The dose selector 4 and housing 3 have complementary markings 20, 21, which provide a visual indication of what discrete dose is set when a user rotates the dose selector 4. The dose selector 4 has a selector peg 22 (shown in FIG. 3) located in line with the visual marking 20, which is arranged to fit between saw-tooth like grooves defined by raised features 23 on an inner surface of the housing 3 (shown in FIG. 4—not all raised features are highlighted), where the bight of each groove is in line with a corresponding visual marking 21 on the housing 3. The selector peg 22 is resiliently deformable, such that as the dose selector 4 is rotated, the selector peg 22 is deformed radially inward each time it travels over a raised feature 23. This arrangement provides some resistance to rotation between the discrete dose settings, and allows the dose setter 4 to “click” into place for each dose.

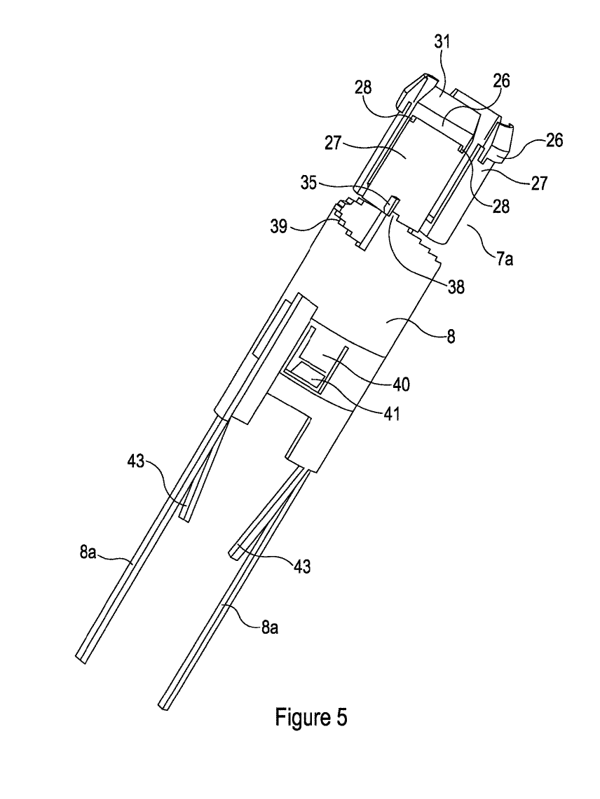

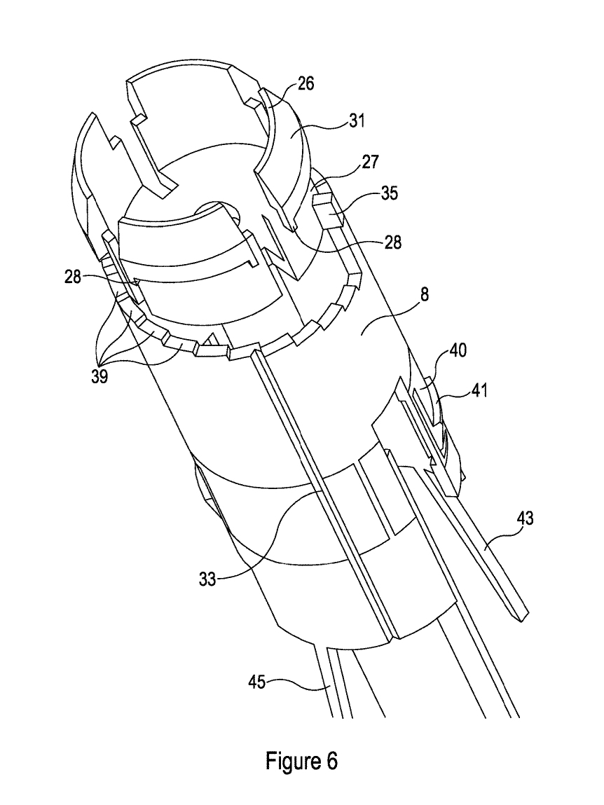

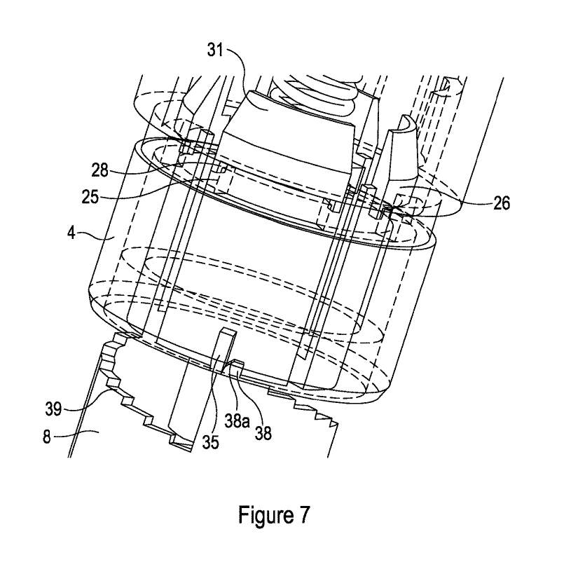

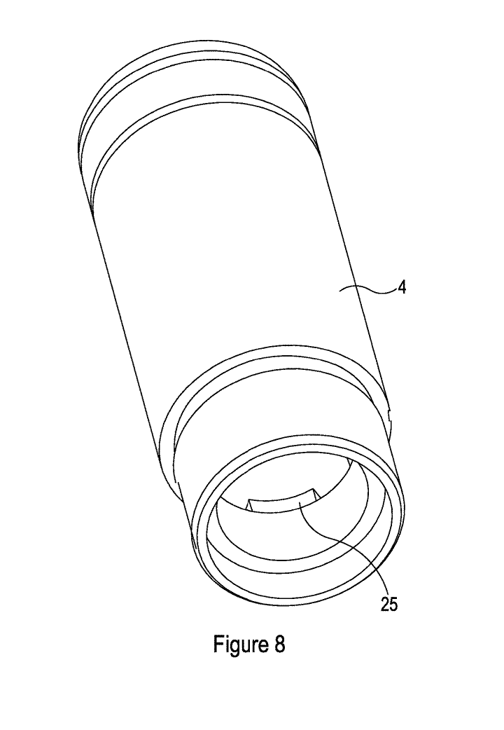

The drive spring 9 is preloaded, and acts to urge the plunger 7 in the proximal direction when the trigger button 5 is pressed. The plunger 7 has a trigger catch 7a (the trigger catch is shown in more detail in FIGS. 5 and 6) at its distal end, which is arranged to co-operate with the trigger button 5. In an initial condition (i.e. before use), the trigger catch 7a is axially held in place relative to the dose selector 4 by four shoulders 25 (see FIGS. 7 and 8) carried by the internal wall of the dose selector 4, which co-operate with four co-operating portions in the form of outwardly projecting teeth 26 on resiliently flexible fingers 27 of the trigger catch 7a. The trigger catch 7a is also rotationally fixed relative to the dose selector, before use, via extended portions 28 which abut the sides of the shoulders 25 (see FIG. 7, where the dose selector 4 has been drawn using dashed lines to help differentiate it from the trigger catch 7a). This ensures that as the dose selector 4 is rotated relative to the housing 3 to set a dose, the trigger catch 7a is also rotated. It will be appreciated by the skilled person that any suitable number of shoulders and co-operating portions may be used, such as three shoulders and three corresponding co-operating potions.

The trigger button 5 has four flexible arms 29 with cam surfaces 30 that co-operate with cam surfaces 31 on the flexible fingers 27 of the trigger catch 7a, such that proximal movement of the trigger button 5 relative to the trigger catch 7a will flex the fingers 27 of the trigger catch 7a radially inward, releasing the outwardly projecting teeth 26 from the shoulders 25. Once the outwardly projecting teeth 26 of the trigger catch 7a have been released from the shoulders 25, the plunger 7 is no longer axially or rotationally coupled to the dose selector 4, and is urged forcefully by the drive spring 9 in the proximal direction.

Once the trigger catch 7a has been released during the above process, the trigger button 5 is prevented from further, significant, proximal movement relative to the dose selector 4 by the ends of the flexible arms of the trigger button 5 abutting the shoulders 25. Alternatively, further co-operating elements can be supplied on the trigger button 5 or housing dose selector 4, or both in order to prevent further movement of the trigger button on release of the trigger catch 7a.

Once the plunger 7 is released, a proximal end 7f of the plunger 7 acts on the bung 16 of the syringe 11. Due to fluid resistance from the liquid 13 in the syringe 11, the load is transferred from the bung 16 to the syringe 11, which transfers the load to the distal barrel portion 10a of the syringe carrier 10. The distal barrel portion 10a of the syringe carrier 10 is then driven forward in the proximal direction. The proximal end of the proximal barrel portion 10b is arranged to be pressed against the user's skin during use (see FIG. 9, which shows the syringe carrier 10 in isolation, distal barrel portion 10a and proximal barrel portion 10b are indicated by two rectangles placed over the syringe carrier). This abutment between the skin and the proximal barrel portion 10b holds the proximal barrel portion 10b in place relative to skin. As the distal barrel portion 10a of the syringe carrier 10 is driven forward in the proximal direction, the compressive connector portion 10c compresses, allowing the needle to extend beyond the syringe carrier 10 and penetrate the user's skin.

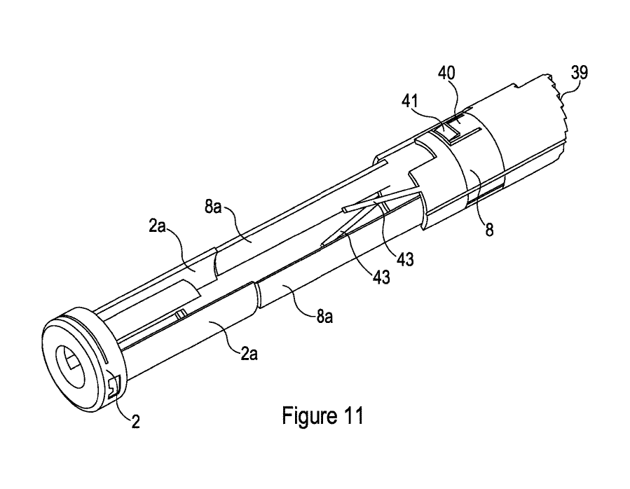

After the needle 15 has traveled a predetermined distance, preferably between about 6-10 mm, the flange 19 of the distal barrel portion 10a of the syringe carrier 10 abuts a stop face 32 on the inner surface of the housing 3 (see FIG. 11), which prevents further proximal movement of distal barrel portion 10a of the syringe carrier 10. The load from the drive spring 9 is now transferred to the bung 16. The load from the drive spring 9 overcomes the fluid resistance of the fluid 13 in the syringe 11, allowing the bung 16 to move proximally through the syringe delivering the fluid to the user through the needle 15 (the medicament delivery phase). Once the dose has been delivered the user pulls the auto-injector 1 away from the skin. This action withdraws the needle 15 and allows the compressive connector portion 10c to expand, allowing the proximal barrel portion 10b of the syringe carrier 10 to cover the exposed needle 15.

In the initial condition, the above process is prevented from occurring by a mechanical interaction between the boot remover 2, lock shuttle 8 and trigger catch 7a. In order for the auto-injector 1 to fire when the trigger button 5 is pressed, the user must first have removed the boot remover 2 and then dialed a dose, in that order. Once these two sequential steps have been completed, the device will fire when the trigger button 5 is pressed. This sequential ordering of steps, described in more detail below, makes it more difficult for the user to accidentally fire the auto-injector 1.

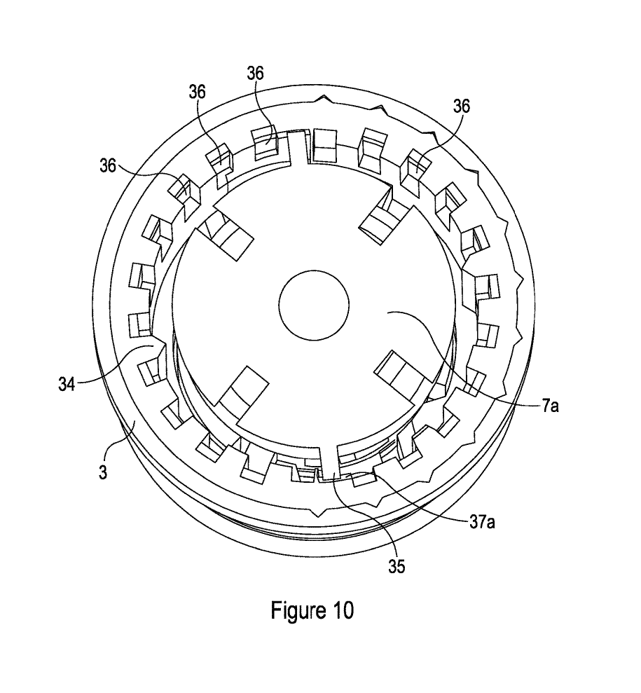

In order to better describe the interaction between the trigger catch 7a and the lock shuttle 8, these components are shown in isolation in FIGS. 5 and 6. The lock shuttle 8 cannot be rotated relative to the housing 3, due to an engagement between an axial track 33 (shown in FIG. 6) running along the outer surface of the lock shuttle 8 and raised guide portion 34 (shown in FIG. 10) running along the inner surface of the housing 3. When unobstructed, the lock shuttle 8 can move axially with respect to the housing 3. In the initial condition, legs 8a of the lock shuttle 8 abut legs 2a of the boot remover 2 (see FIG. 11 which shows the boot remover 2 and lock shuttle 8 in isolation). This abutment prevents the lock shuttle 8 moving in the proximal direction while the boot remover 2 is still attached to the auto-injector 1. This restriction in proximal movement of the lock shuttle 8 also prevents rotation of the dose selector 4, meaning a dose cannot be set (described in more detail below). When the user removes the boot remover 2, the legs 2a no longer abut the legs 8a, and the lock shuttle 8 is free to move in the proximal direction. The boot remover 2 may be attached to the housing 3 via any suitable means, such as, for example, interlocking features on the housing and boot remover 2.

The trigger catch 7a has a pair of pegs 35 located on either side of its external surface. These pegs are arranged to fit within tracks 36 located on an inner surface of the housing 3 (see FIG. 4—not all tracks have been referenced for clarity). Each track 36 corresponds with a specific dose setting and has a length which corresponds with the distance the plunger 7 needs to travel in order to deliver the correct dose (i.e. a smaller dose corresponds with a shorter track and a larger dose corresponds with a longer track). For each specific dose, there are two corresponding tracks having the same length located on either side of the housing 3. This is to accommodate the two pegs 35 on either side of the trigger catch 7a. Therefore, the dose selector 4 may be rotated through less than 180 degrees between no dose being set, and a maximum dose being set. However, the skilled person will realise that only one peg 35 and one track may be used. As the auto-injector 1 is fired, the pegs 35 travel along a specific track 36 corresponding with a specific dose. When the pegs 35 reach the end of the tracks 36, they abut raised surfaces 36a within the tracks which prevent further proximal movement of the plunger 7.

By rotating the dose selector 4 to a specific dose, the pegs 35 can be made to line up with the corresponding specific track (i.e. lined up when looked at end on). The tracks 36 are separated by internally raised portions 37. In the initial condition, each peg 35 is not lined up with a track 36, but instead lined up with a pair of shoulders 37a on the inner surface of the housing 3 that correspond with no dose being set. Therefore, the auto-injector 1 will not fire prior to a dosage being selected, due to abutment of the pegs 35 with the shoulders 37a on the inner surface of the housing 3.

In the initial condition the dose selector cannot be rotated anti-clockwise to set a dose due to abutment of the pegs 35 with a pair of dial locks 38 located on the distal end of the lock shuttle 8 (see FIG. 5 which shows one of the dial locks 38, the other being located on the opposite side of the lock shuttle 8). The dial locks 38 extend past the pegs 35 so as to prevent anti-clockwise rotation. The dose selector 4 is also initially prevented from rotating clockwise due to abutment between a rib 53 on the inner surface of the housing 3 and the peg 35 (see FIG. 15, which shows a close up view of the peg 35 with the housing 3 partially transparent so as to show the rib 53). FIG. 15 also shows a ridge 54 on the inner surface of the housing 3, which provides an initial resistance to rotating the dose selector 4 in the anti-clockwise direction. This helps to prevent accidentally setting a dose during handling once the boot remover 2 has been removed. FIG. 15 also shows the peg 35 having a tapered like profile 35a. This arrangement assists the peg 35 in travelling past the ridge 54 when a user rotates the dose selector 4 to set a dose. In order to select a dose using the dose selector 4 the dial locks 38 must be proximally displaced such that they are no longer in the rotational path of the pegs 35. However, in the initial condition, the boot remover 2 prevents axial displacement of the lock shuttle 8 and hence the dial locks 38. Therefore, the boot remover 2 must be removed in order to allow proximal movement of the lock shuttle 8. The dial locks 38 feature a chamfered edge 38a, such that when the boot remover 2 has been removed and the dose selector 4 is rotated, the pegs 35 interact with the chamfered edge 38a to help proximally displace the dial lock 38 out of the rotational path of the pegs 35. This then allows the user to continue to rotate the dose selector 4 to a specific dose. Therefore, the dose selector 4 cannot be rotated prior to removal of the boot remover 2.

This sequential ordering of steps prevents a user from accidentally firing the device, forcing the user to first remove the boot, and then set a dose.

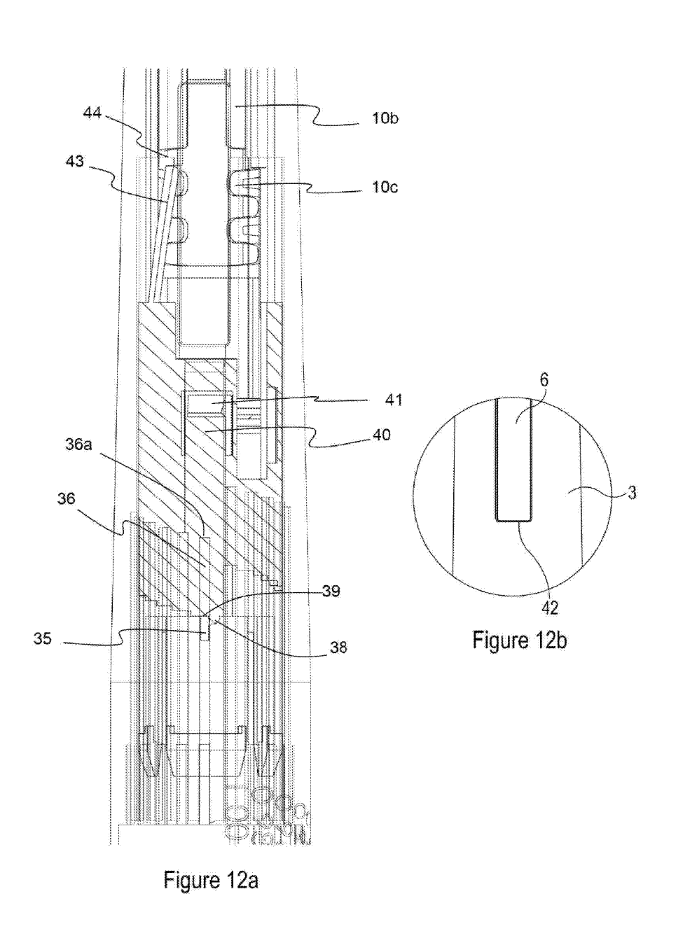

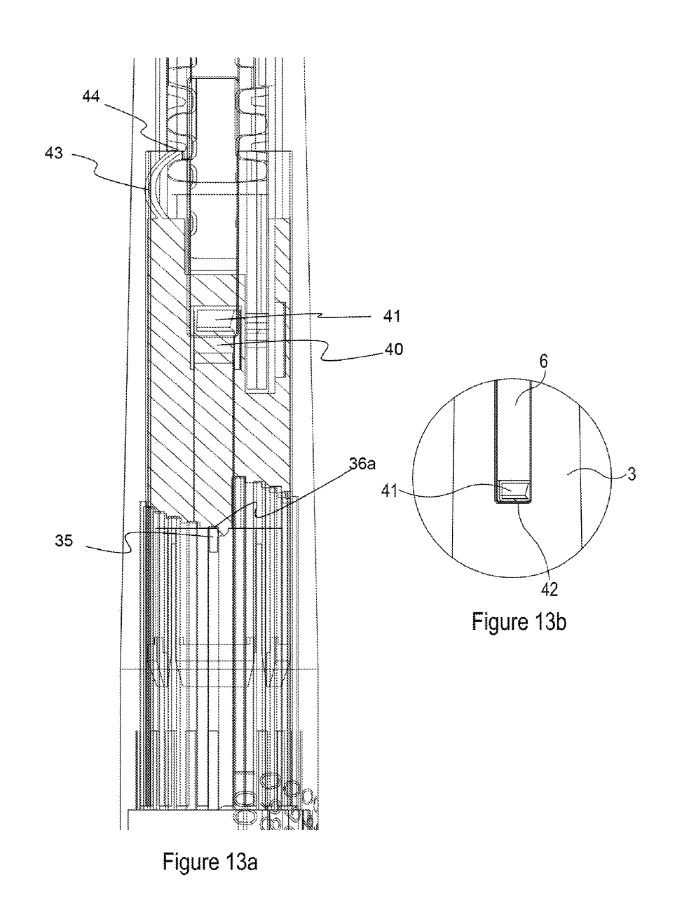

The lock shuttle 8 is arranged to travel the same distance in the proximal direction during firing of the auto-injector 1, regardless of what dose has been set. This is facilitated by the lock shuttle 8 having a series of steps 39 (see FIGS. 5, 6, and 7 which show some of the steps 39—for clarity, not all steps are referenced) along its distal end defining different lengths of the lock shuttle 8, where each step 39 corresponds with a specific track 36 (and therefore dose) (see FIGS. 12a and 13a which show a transparent view of the housing 3, along with the trigger catch 7a and the lock shuttle 8). As described above, in the embodiment shown, there are two pegs 35 on opposite sides of the trigger catch 7a. Therefore, there are two corresponding steps 39 for each dose, one located on either side of the lock shuttle 8. Once the user has rotated the dose selector 4 to a desired dose, the pegs 35 line up with the corresponding steps 39 on either side of the lock shuttle 8 and the corresponding tracks 36 on either side of the housing 3 for that dose. As the auto-injector 1 is fired, the plunger 7 is axially displaced in the proximal direction under the force of the drive spring 9. The lock shuttle 8 is not substantially proximally displaced until the pegs 35 make contact with the corresponding steps 39 relating to the dose that has been selected. Once the pegs 35 abut the steps 39, the lock shuttle 8 is also axially displaced in the proximal direction under the force of the drive spring 9, until the pegs 35 reach the end of the tracks 36 and abut the raised surfaces 36a within the tracks 36. The differing height of the steps 39 therefore provide different offsets which the pegs 35 must travel before coming into contact with, and displacing, the lock shuttle 8, i.e. the length of the lock shuttle 8 as seen from the pegs 35 is differs depending on what step 39 is selected.

The arrangement of the relative lengths of the tracks 36 and the offset provided by the corresponding step 39 is such that the lock shuttle 8 is driven by the drive spring 9 for substantially the same distance no matter what dose has been set prior to firing. For example, if the user rotates the dose selector 4 to the first dose setting (0.80 ml) shown in FIG. 1, the pegs 35 line up with relatively short tracks 36 and also line up with steps 39 which define a relatively short offset, and hence longer length of the lock shuttle 8. In some embodiments, the smallest dose may have a step 39 that immediately contacts the peg 35 after the dose has been set, i.e. there is no gap between the peg 35 and the first step 39, and so the offset is effectively zero. This is shown in FIG. 12a which shows a close up of the housing 3 mid-delivery (where the housing 3 is shown as partially transparent), and where the smallest dose has been set. As the auto-injector 1 is fired, the pegs 35 of the plunger 7 begin to travel down the tracks 36, and due to the abutment of the pegs 35 with the steps 39, the lock shuttle 8 is also driven proximally forward with the plunger 7. The plunger 7 reaches the end of its travel when the pegs 35 abut the raised surfaces 36a within the tracks 36 (shown in FIG. 13a).

If a larger dose is set, the pegs 35 will line up with relatively long tracks and also line up with steps which define a relatively long offset, and hence shorter length of the lock shuttle 8. When the auto-injector 1 is fired, the pegs 35 begin to travel down the longer tracks 36, and due to the relatively short length of the lock shuttle 8 as defined by the offset of the steps 39, the pegs 35 will travel a further distance before coming into contact with the steps 39.

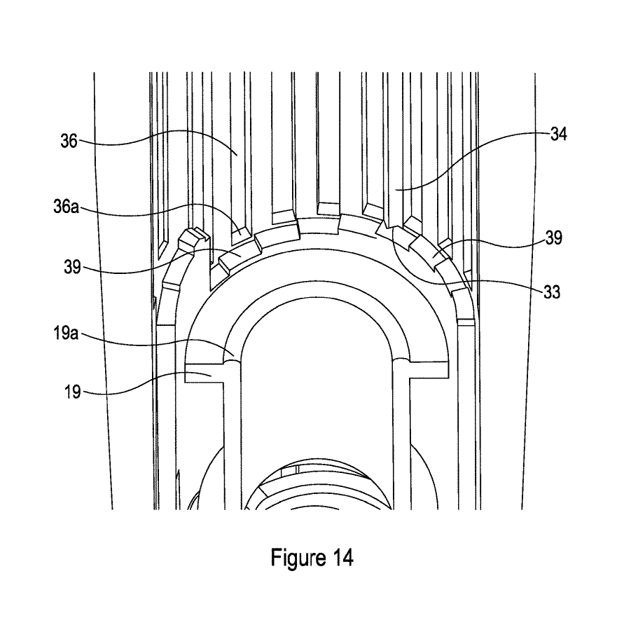

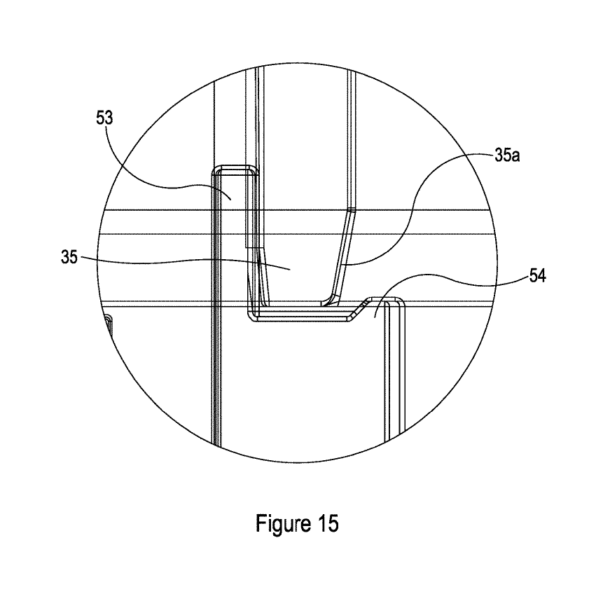

The relative change in height (offset) of each step 36 corresponds with the relative change in the length of each track 36. This is shown in FIG. 14 which shows an isolated section of the inside of the housing 3 with the lock shuttle 8 fully driven proximally forwards. As can be seen, the end 36a of each track 36 is at substantially at the same axially point as its corresponding step 39. Note that FIG. 14 does not show the syringe for clarity.

The lock shuttle 8 features a mechanism for providing an indication to a user that the full dose has been delivered. The lock shuttle 8 features a pair of lock arms 40 located on either side of the lock shuttle 8. Each lock arm 40 has a wedge portion 41 (see FIGS. 5 and 6), which, in the initial condition, press against an inner surface of the housing 3. This arrangement bends each lock arm 40 radially inward, creating tension in the lock arms 40. Once the lock shuttle 8 has traveled its full length after firing, the wedge portions 41 line up with the viewing windows 6 on either side of the housing 3. The viewing windows are in the form of cut outs in the housing 3. When the wedge portions 41 reach the viewing windows 6, the wedge portions 41 no longer press against the inner surface of the housing 3 (due to the presence of the cut out), and so the tension stored in the lock arms 40 is released, snapping the wedge portions 41 into the cut outs of the viewing windows 6. Abutment of the wedge portions 41 with a distal surface 42 of the cut out prevents the lock shuttle from moving distally once the device has been fired. This provides both a visual indication to the user that the device 1 has been fired, and an audio indication as the wedge portions 41 “click” into place. The wedge portions 41 may have a symbol or coloured portion to help easily identify the wedge potions 41 when they are located in the viewing windows 6. FIG. 12b shows a close up of one of the viewing windows 6 before the auto-injector 1 has been fired, and FIG. 13b shows the viewing window 6 after the auto-injector 1 has been fired, with the wedge portion 41 showing through the viewing window 6. In other words, regardless of the specific dose delivered, the lock shuttle 8 ends up in the same final position such that the wedge portions 41 line up with the viewing windows 6. This then gives the user an indication that the full does has been delivered.

In order to prevent the wedge portion 41 from prematurely entering the cut-out prior to firing (for example, if the user orientates the auto-injector 1 such that the proximal end faces the ground after the boot remover 2 has been removed), a pair of flexible positioning arms 43 are provided on either side of the proximal end of the lock shuttle 8, and which are arranged to abut a lip 44 around the inner surface of the housing 3. The flexible positioning arms 43 extend from the lock shuttle 8 at an angle offset from the axis of the auto-injector 1. As the lock shuttle 8 moves proximally forward, abutment between the flexible positioning arms 43 and the lip 44 occurs before the wedge portions 41 reach the viewing windows 6. The flexible positioning arms 43 have sufficient rigidity so as to not significantly deform when they abut the lip 44, unless the lock shuttle is being driven by the drive spring 9. When the lock shuttle 8 is proximally driven by the drive spring 9 during firing, the flexible positioning arms 41 are deformed against the lip 44 under the force of the drive spring 9, which then allows the wedge portions 41 to reach the viewing windows 6 and snap into place. FIG. 12a shows one positioning arm 43 prior to firing (un-deformed) and FIG. 13a shows the positioning arm 43 in a deformed state after firing. This arrangement negates the need for positioning springs and complex geometry on the inner surface of the housing 3 in order to prevent the lock shuttle 8 from sliding proximally forward prior to firing.

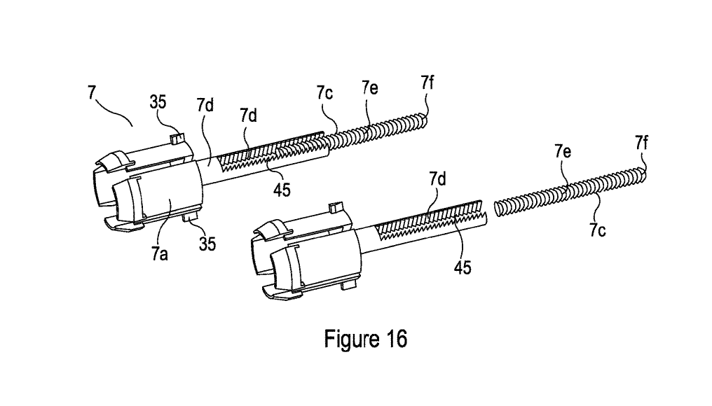

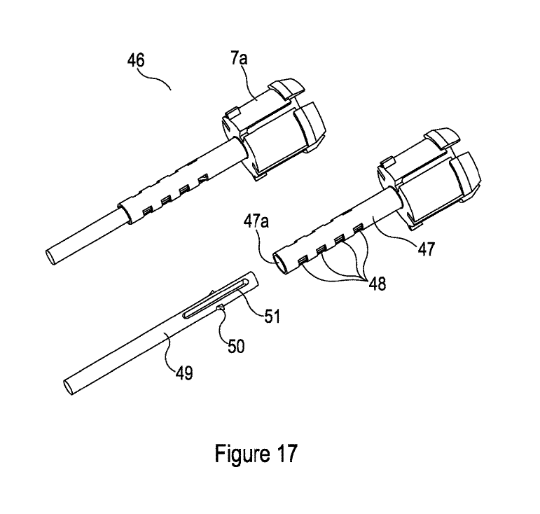

The plunger 7 will now be described in more detail with reference to FIGS. 16 and 17. FIG. 16 show two perspective views of the plunger 7 in an assembled state and unassembled state. The plunger 7 comprises the trigger catch 7a, a plunger stem 7b, and an extended plunger portion 7c. The trigger catch 7a and the plunger stem 7b are integrally formed. The extended plunger portion 7c is formed separately from the trigger catch 7a and the plunger 7b. The stem 7b features a recessed region 45 along a part of its length. The recessed region 45 is arranged to receive and accommodate the extended plunger portion 7c. The recessed region 45 couples with the extended plunger portion 7c via a series of interlocking saw tooth features 7d located on the inner surface of the recessed region 45 and saw tooth features 7e located on the outer surface of the extended plunger portion 7c. During manufacture of the plunger 7, the extended plunger portion 7c is placed at a predetermined position within the stem 7b such that the total length of the plunger 7 is the desired length for the purpose of the plunger 7. The extended plunger portion 7c may be fixed in position, using, for example, an adhesive. Alternatively, there may be sufficient friction between the saw tooth features 7d, 7e such that adhesive is not required. A proximal end 7f of the extended plunger portion 7c is arranged to contact a bung of a cartridge.

Advantageously, by providing a plunger that is formed from two separate component parts, the length of the plunger 7 may be easily adjusted during manufacture of the plunger 7. During production of the plunger 7, two moulds are required; one for the trigger catch 7a and stem 7b, and one for the extended plunger portion 7c. However, the dimensions of these two separate component parts do not need to be changed to produce plungers of different lengths. Therefore, moulds used to produce the trigger catch 7a and stem 7b, and extended plunger portion 7c do not need to be reconfigured each time the length of the plunger needs to be adjusted. Instead, a desired length of the plunger 7 can be achieved by inserting the extended plunger portion 7c in the stem 7b at a desired location, such that the total length of the plunger 7 corresponds with the desired length. Furthermore, the saw-tooth nature of the coupling between the stem 7b and the extended plunger portion 7c allows discrete lengths to be easily chosen.

FIG. 17 show a modified plunger 46 according to an alternative embodiment. The modified plunger 46 comprises a trigger catch 7a, a stem 47 featuring four pairs of apertures 48 arranged on either side of the length of the stem 47, and an extended plunger portion 49. The extended plunger portion 49 features a pair of wedges 50 which are located on either side of the extended plunger portion 49 and which are arranged, when in a relaxed position, to extend radially outwardly into a pair of apertures 48 on the stem 47. The extended plunger portion 49 also features a hollowed out portion 51 located between the wedges 50, which is arranged to allow the extended plunger portion 49 to be radially compressed, i.e. forcing the wedges 50 together causes the extended plunger potion to compress into the hollowed out portion 51. The material of the extended plunger portion 49 is such that, on removal of the force, the extended plunger potion 49 returns to its relaxed state. In order to set the length of the plunger 46, the extended plunger portion 49 is loaded into a proximally facing opening 47a on the stem 47. Prior to insertion, the wedges 50 are compressed radially inward such that the wedges 50 do not prevent the extended plunger portion 49 from entering the opening 47a. Once the extended plunger portion is located in the desired position, the wedges 50 are released, allowing the hollowed out portion 51 to return to their relaxed position such that they enter a pair of apertures 48, locking the extended plunger portion 49 in place relative to the stem 47. It will be appreciated that any number of apertures 48 may be used, and that it is not necessary for there to be a pair on either side, i.e. there may be one aperture per length setting.

The embodiments shown in FIGS. 16 and 17 allow a syringe plunger length to be adjusted during assembly by altering the position at which two components of the plunger are assembled. This allows plunger components of fixed lengths to be manufactured that can then be used in a variety of different devices, such as the auto-injector 1, which can accept a wide range of syringe fill volumes, for example, from 0.02 ml to 1.0 ml.

Since the plunger 7 can be tailored to a particular device having a particular syringe fill volume, a further advantage of the plunger described herein is that a gap between the bung 16 and the plunger end 7f, prior to firing the device, can be reduced. This reduces the velocity at which the plunger 7 strikes the bung reducing resultant glass stresses on the syringe at the moment of impact. In an embodiment, the length of the plunger 7 may variable such as to be usable with syringe volumes that vary in 0.01 ml increments.

With reference to FIGS. 18 to 23, there will now be described a damping arrangement which helps to reduce the magnitude of the peak impacts during a firing operation of the auto-injector 1.

As described above, once the trigger catch 7a is released, the first 10 mm or so of travel drives the needle 15 into the skin (the needle insertion phase). There are typically two main impacts during this phase. The first peak impact occurs when the plunger end 7f makes contact with the bung 16 of the syringe 11. The load from the plunger 7 is applied to the bung 16, but resistance to compression from the fluid 13 in the syringe 11 means no fluid is expelled and instead the syringe 11 and syringe carrier 10 are driven proximally forward (as described above). The second peak impact occurs when the syringe carrier 10 reaches the end of its travel by abutting the stop face 32. The stop face 32 may be in the form of a lip around, or protrusion from, the inner surface of the housing 3.

These peak impacts can damage the syringe 11, which is usually made from a brittle material such as glass or plastic material. The following embodiments help to reduce the magnitude of these peak impacts.

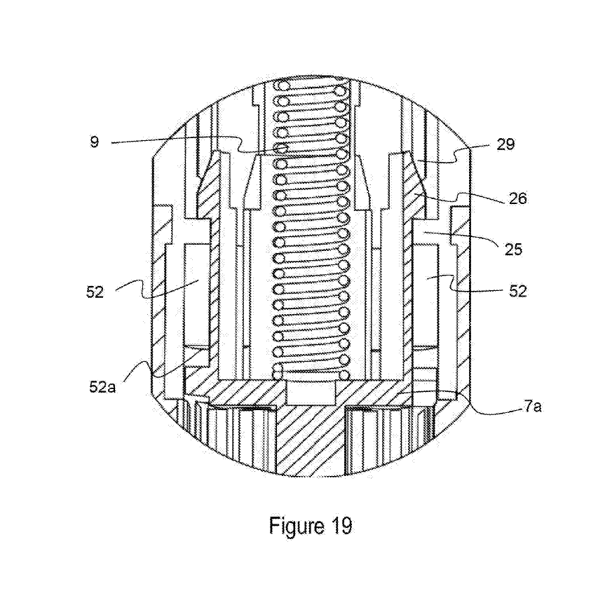

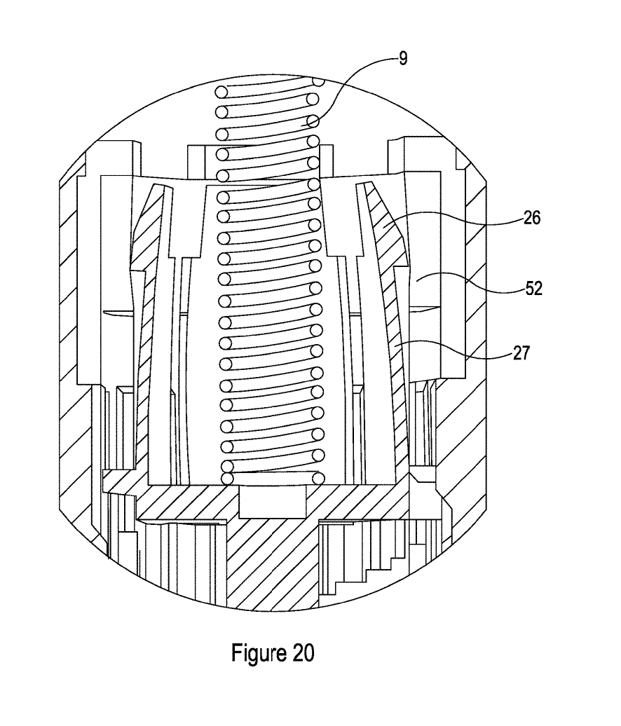

A rubber insert 52 is arranged on an inner surface of the dose selector 4 below the shoulder 25 (see FIGS. 19 and 20). The rubber insert 52 provides a relatively high frictional surface compared with the typical plastic like material used to construct an auto-injector (such as POM Acetal). The rubber insert 52 may comprise TPE rubber. FIG. 19 shows a close up cross sectional view of the auto-injector 1 in the initial condition, where the trigger catch 7a is axially fixed due to the abutment of the outwardly projecting teeth 26 with the shoulders 25. FIG. 20 shows a close up cross sectional view of the auto-injector 1 just after the trigger button 5 has been pressed, releasing the outwardly projecting teeth 26 from the shoulders 25 and allowing the plunger 7 to be displaced proximally under the force of the drive spring 9. Once the outwardly projecting teeth 26 on the flexible fingers 27 have cleared the shoulders 25, the outwardly projecting teeth 26 slide against the rubber insert 52 (see FIG. 20). The friction between the outwardly projecting teeth 26 and the rubber insert 52 helps to reduce the acceleration of the plunger 7 during the needle insertion phase. The inner diameter of the rubber insert 52 is smaller than the outer diameter of the outwardly projecting teeth 26 when the resiliently flexible fingers 27 are in a relaxed (un-flexed) position. This arrangement means that as the outwardly projecting teeth 26 pass through the rubber insert 52, the resiliently flexible fingers 27 are held radially inward. This provides a radially outward biasing force on the outwardly projecting teeth 26, increasing the friction between the teeth 26 and the rubber insert 52. FIG. 20 shows the rubber insert 52 undergoing deformation as the teeth 26 slide past it due to the outward biasing force.

The axial length of the rubber insert 52 is arranged such that the outwardly projecting teeth 26 reach a proximal end 52a of the rubber insert 52 just after the syringe carrier 10 has reached the end of its travel. This leads to the needle insertion phase being damped, and the medicament delivery phase being substantially un-damped by the rubber insert 52. This arrangement reduces the load delivered to the bung 16, and hence the magnitude of the first peak impact, during the needle insertion phase by reducing the plunger's 7 acceleration under the force of the drive spring 9. This arrangement also reduces the magnitude of the second peak impact as the syringe carrier 10 will not be travelling as fast it would otherwise have been when the flange 19 of the syringe carrier 10 abuts the stop face 32. Once the needle insertion phase has been complete (when the flange 19 of the syringe carrier 10 abuts the stop face 32), the outwardly projected teeth 26, which continue to be displaced proximally under the force of the drive spring 9, clear the proximal end of the rubber insert 52a allowing the fluid delivery phase to commence un-damped. The skilled person will recognise that any material may be used for the rubber insert 52 that provides a relatively high friction surface to help slow the acceleration of the plunger. In alternative embodiments, the high friction surface may be applied instead, or in addition, to the teeth 26.

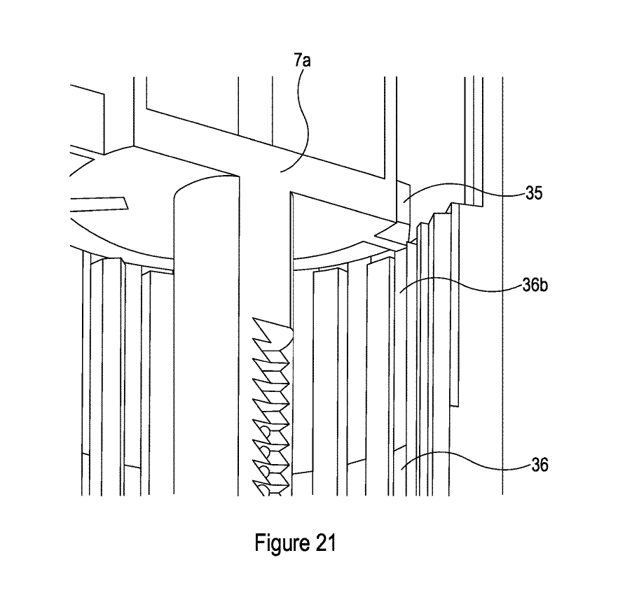

Any suitable surface may be used to apply the relatively high friction surface. In an embodiment, each track 36 features a separate rubber insert 36b (see FIG. 21). The rubber inserts 36b in the tracks 36 are arranged to provide a relatively high frictional surface for the pegs 35. The axial length of the rubber inserts 36b are such that the pegs 35 reach the a proximal end of the rubber inserts 36b just after the syringe carrier 10 has reached the end of its travel, meaning that the fluid delivery phase is substantially un-damped by the rubber inserts 36b. The separate rubber inserts 36b in the tracks 36 may be used separately, or in combination with the rubber insert 52.

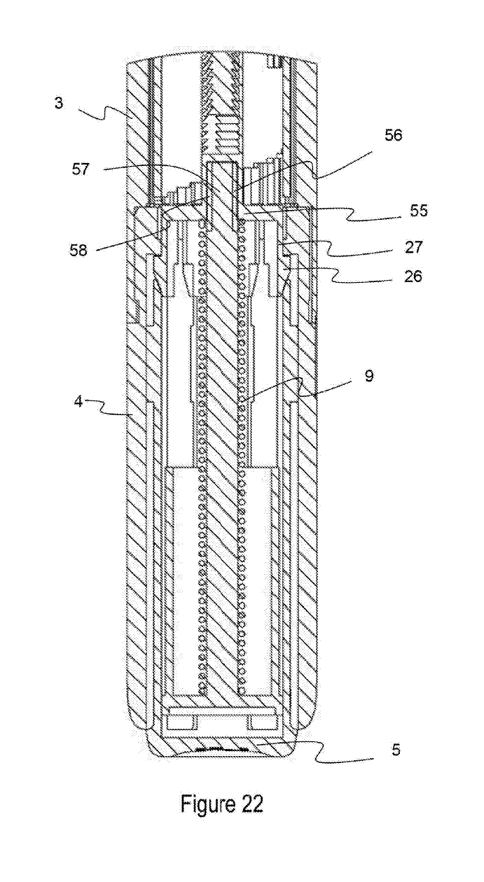

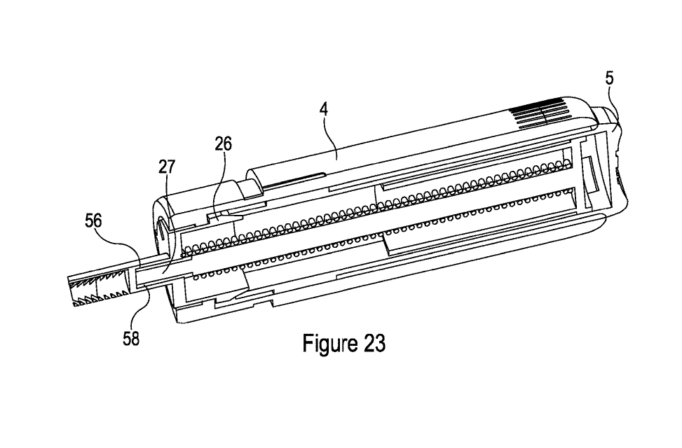

FIGS. 22 and 23 show the auto-injector 1 having a modified plunger 55 according to another embodiment. FIG. 22 shows a cross sectional of the distal end of the auto injector 1, and FIG. 23 shows a cross section perspective view of the distal part of the auto injector 1 without the housing 3. The modified plunger 55 operates in the same way as the plunger 7, but is modified to include a central bore 56 which is arranged to receive a rod 57. The rod 57 extends through the drive spring 9 and is axially fixed with respect to the dose selector 4. The rod 57 comprises a high friction surface 58, the dimensions of which are arranged so as to achieve an interference fit between the bore 56 and rod 57. In the initial, unfired, condition, the rod 57 is located within the bore 56. As the auto-injector 1 is fired, the plunger 55 moves axially with respect to the rod 57. As the rod 57 does not move axially relative to the dose selector 4 (and housing 3), the inner surface of the bore 56 slides against the high friction surface 58 of the rod 57, which slows the acceleration of the plunger 55. The axial distance at which the rod 57 penetrates the bore 56 is arranged such that the rod 57 exits the bore 56 just after the syringe carrier 10 has reached the end of its travel. This arrangement allows the rod 57 to exit the bore 56 once the needle insertion phase has ended, which removes the damping effect and allows the medicament delivery phase to begin, undamped.

The high friction surface 58 may comprise a rubber material. The skilled person will appreciate that the bore 56 may carry the high friction surface instead of, or as well as, the rod 57.

In an embodiment, the high friction surface 58 comprises four arms which extend radially outward (not shown) in a cross like manner when viewed end on. The distance between the end of each arm is arranged such that the distance is larger than the diameter of bore 56 of the plunger 55. This arrangement leads to the arms deforming when the rod 57 is inserted into the bore 56, which helps create the interference fit between the rod 57 and bore 56. The skilled person will realise that any suitable shape of high friction material may be used to achieve an interference fit.

The second peak impact is generated when the syringe carrier 10 hits the stop face 32 and transfers load from the syringe carrier 10 to the syringe 11. While the syringe carrier 10 comes to an abrupt halt, the plunger 7, under the force of the drive spring 9, continues to apply pressure to the bung 16. Fluid resistance initially prevents the bung 16 from forward movement, which leads to a slight compression of the bung 16 and which transfers load through a wall of the syringe 11 as the bung 16 tries to expand. This transfer of load can temporarily overcome the interference fit between the syringe 11 and the syringe carrier 10, driving the syringe 11 proximally forward within the syringe carrier 10 by a small distance (typically around 0.5 mm-1 mm), until the fluid 13 beings to flow from the needle 15. As the syringe 11 moves proximally relative to the syringe carrier 10, the distance between the flange 19 of the syringe carrier 10 and the flange 14 of the syringe 11 is reduced and the resiliently deformable flange damper 18, which is arranged between the flanges 19, 14, is compressed (see FIG. 18 which shows the resiliently deformable flange damper 18 in an uncompressed state). Compression of the resiliently deformable flange damper 18 helps to dissipate this load, thereby reducing stress on the syringe flanges 14. The resiliently deformable flange damper 18 may comprise rubber, or any other resiliently deformable material, and may comprise any shape suitable for reducing load of the syringe flanges 14. For example, FIG. 18 shows the resiliently deformable flange damper 18 having a raised lip 18a to aid with dissipating the load.

Embodiments of the invention have been described. Variations and modifications will suggest themselves to those skilled in the art without departing from the scope of the inventions as defined by the appended claims. Furthermore, separate embodiments that have been described may be combined with other embodiments described, or used separately.

While various parts have been referred to as shoulders, lips, pegs, protrusions, tracks, it will be appreciated that these features may be replaced by features which achieve the same effect, i.e. a single lip around an inner surface may be replaced by one or more shoulders of other protrusions. Furthermore, where cooperating features between the housing and the plunger or lock shuttle have been described, such as the positioning arms and lip, it will be understood that these may be swapped around where appropriate. For example, the positioning arms 43 may be fixed to the housing 3 and the lip 44 arranged around an outer surface of the lock shuttle 8. The boot 2b has been described as being separate from the boot remover 2, however the boot remover 2 may be integral with the boot 2b, and the leg 2a of the boot remover 2 may be replaced by an extended portion of the boot 2b.

1-44. (canceled)

45. An injection device for delivering a medicament from a container, the device comprising:
a housing for housing a container;
a plunger substantially housed within the housing and which is movable within the housing;
a force applicator for applying a force to the plunger;
a trigger coupled to the force applicator for releasing the force applicator to fire the device;
a boot;
a dose selector for allowing a user to select a dose of medicament; and
a first mechanical interlock arranged such that the device cannot be fired until the dose selector has been operated to select the dose of medicament; and
a second mechanical interlock arranged such that the dose selector cannot be operated to select the dose of medicament prior to removal of the boot.

46. An injection device according to claim 45, wherein the first mechanical interlock is provided by a first coupling between the housing and the plunger.

47. An injection device according to claim 46, wherein the first coupling comprises an abutment between a first abutment element on, or mechanically coupled to, one of the housing and the plunger and a second abutment element on, or mechanically coupled to, the other of the housing and the plunger.

48. An injection device according to claim 47, wherein the first mechanical interlock is arranged such that the abutment of the first abutment element and the second abutment element prevents the plunger from being displaced axially.

49. An injection device according to claim 45, wherein the second mechanical interlock is provided by a second coupling between the dose selector and the boot.

50. An injection device according to claim 49, wherein the second coupling comprises an abutment between a third abutment element on, or coupled to, the dose selector and a fourth abutment element on, or coupled to, the boot.

51. An injection device according to claim 45, wherein the dose selector is rotatable relative to the housing, said rotation allowing a user to select the dose of medicament.

52. An injection device according to claim 51, wherein the dose selector and plunger are rotationally coupled such that rotation of the dose selector rotates the plunger and removes the abutment of the first abutment element and the second abutment element.

53. An injection device according to claim 47, wherein the first abutment element is either a peg or shoulder on the plunger and the second abutment element is the other of a peg or shoulder on the housing.

54. An injection device according to claim 52, wherein the second mechanical interlock is arranged such that the abutment of the third abutment element and the fourth abutment element prevents the dose selector from being rotated relative to the housing.

55. An injection device according to claim 54, wherein removal of the boot removes the second coupling between the third abutment element and the fourth abutment element.

56. An injection device according to claim 50, wherein the third abutment element is a first surface on the plunger and the fourth abutment element is a second surface mechanically coupled to the boot.

57. An injection device according to claim 56, further comprising a sleeve housed in the housing and which is rotationally fixed and axially moveable with respect to the housing.

58. An injection device according to claim 57, wherein the sleeve comprises the second surface and wherein the sleeve is axially coupled to the boot such that axially movement of the sleeve is restricted while the boot is attached to the device.

59. An injection device according to claim 57, further comprising a boot remover for removing the boot, and wherein the boot remover abuts the sleeve while the boot remover is attached to the device, preventing axial movement of the sleeve.

60. An injection device according to claim 47, wherein the second coupling comprises an abutment between a third abutment element on, or coupled to, the dose selector and a fourth abutment element on, or coupled to, the boot, and wherein the first abutment element and the third abutment element are the same abutment element.

61. An injection device according to claim 57, wherein, when the device is fired, the plunger is arranged to abut the sleeve so as to axially displace the sleeve, and wherein the abutment between the plunger and the sleeve is via the first and/or third abutment element abutting a distal surface of the sleeve.

62. An injection device according to claim 57, wherein the housing comprises a viewing window, and wherein the sleeve is arranged such that a portion of the sleeve is visible through the viewing window after the device has been fired.

63. An injection device according to claim 57, wherein the sleeve comprises a step like profile along its distal end, where at least one step corresponds with a particular dose.

64. An injection device according to claim 63, wherein the steps provide the distal surface that the first and/or third abutment element abut, and
wherein the sleeve comprises a resiliently flexible arm having a wedge portion, and is arranged such that, prior to firing the device, the resiliently flexible arm is bent radially inward due to an abutment between the wedge portion and an inner surface of the housing, placing the resiliently flexible lock arm under tension, and is further arranged such that after firing the injection device, the wedge portion is proximally displaced so as to line up with the viewing window such that the wedge portion no longer abuts the inner surface of the housing, allowing the tension in the resiliently flexible lock arm to be released, driving the wedge portion into the viewing window.
